(19) United States
(12) Patent Application Publication
(10) Pub. No.: US 2009/0149469 A1
(43) Pub. Date: Jun. 11, 2009

(54) Phenyl-[4-(3-phenyl-1h-pyrazol-4-yl)-pyrimidin-2-yl]-amine derivatives as igf-ir inhibitors

(76) Inventor: Carlos Garcia-Echeverria, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/584,828

(22) PCT Filed: Jan. 7, 2005
(86) PCT No.: PCT/EP05/00093

§ 371 Date: Jun. 27, 2006

Related U.S. Application Data
(60) Provisional application No. 60/535,626, filed on Jan. 9, 2004.

(51) Int. Cl. A61K 31/505; C07D 239/42; A61K 31/496; A61P 9/00; A61P 35/00

(52) U.S. Cl. 514/252.19; 544/297; 514/275

(57) ABSTRACT

The present invention relates to a compound of formula I

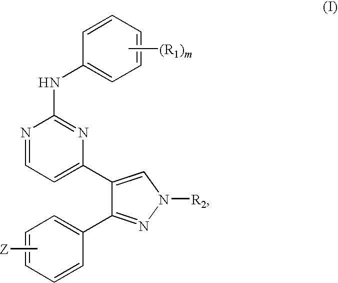

and derivatives thereof. Furthermore, the invention relates to the use of such compounds as medicaments. In addition, the invention relates to the use of such compounds for manufacturing a medicament useful for treating proliferative diseases.

The invention relates to phenyl-[4-(3-phenyl-1H-pyrazol-4-yl)-pyrimidin-2-yl]-amine derivatives and pharmaceutical compositions comprising such derivatives and to the use of such derivatives—alone or in combination with one or more other pharmaceutically active compounds—for the preparation of pharmaceutical compositions for the treatment especially of a proliferative disease, such as a tumour.

The invention relates to compounds of formula I

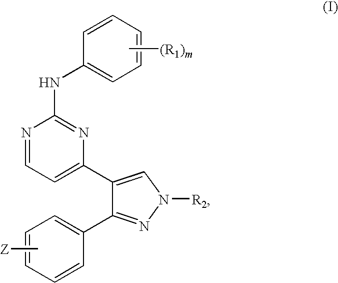

wherein

m is from 1 to 5;

R₁ is lower alkyl-sulfonyl; unsubstituted, mono- or di-substituted amino-sulfonyl; unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;

or two vicinal R₁ substituents together with the phenyl carbon atoms to which they are attached form a heterocyclic ring;

R₂ is hydrogen, unsubstituted or substituted lower alkyl or a heterocyclic radical; and

Z is benzyloxy;

or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

The general terms used hereinbefore and hereinafter preferably have within the context of this disclosure the following meanings, unless otherwise indicated:

Where the plural form is used for compounds, salts, and the like, this is taken to mean also a single compound, salt, or the like.

Asymmetric carbon atoms of a compound of formula I that are optionally present may exist in the (R), (S) or (R,S) configuration, preferably in the (R) or (S) configuration. Substituents at a double bond or a ring may be present in cis- (=Z-) or trans (=E-) form. The compounds may thus be present as mixtures of isomers or preferably as pure isomers.

Preferably alkyl contains up to 20 carbon atoms and is most preferably lower alkyl.

The prefix “lower” denotes a radical having up to and including a maximum of 7, especially up to and including a maximum of 4 carbon atoms, the radicals in question being either unbranched or branched with single or multiple branching.

Lower alkyl is, for example, methyl, ethyl, n-propyl, isopropyl, n-butyl, isobutyl, sec-butyl, tert-butyl, n-pentyl, isopentyl, neopentyl, n-hexyl or n-heptyl.

Lower alkyl R₂ is preferably methyl, ethyl or isopropyl, most preferably methyl.

Substituted lower alkyl is lower alkyl as defined above where one or more substituents may be present, such as amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, amidino, guanidino, ureido, mercapto, lower alkylthio, halogen or a heterocyclic radical.

Substituted lower alkyl R₂ is preferably lower alkyl substituted by N,N-di-lower alkylamino or lower alkyl-piperidyl.

Mono- or di-substituted amino-sulfonyl is amino-sulfonyl, wherein the amino group is substituted by one or two radicals selected independently of one another from e.g. unsubstituted or substituted lower alkyl or a heterocyclic radical.

Unsubstituted, mono- or di-substituted amino-sulfonyl R₁ is preferably unsubstituted amino-sulfonyl.

Mono- or di-substituted amino is amino substituted by one or two radicals selected independently of one another from e.g. unsubstituted or substituted lower alkyl or a heterocyclic radical.

Mono- or di-substituted amino R₁ is preferably N-lower alkylamino or N,N-di-lower alkylamino, respectively.

Mono- or di-substituted amino R₄ is preferably N-lower alkylamino or N,N-di-lower alkylamino, respectively.

R₄-lower alkyl-X—, wherein R₄ is halogen, includes that the lower alkyl moiety of R₄-lower alkyl-X— is substituted with more than one halogen atom, i.e. with up to three halogen atoms, and is preferably trifluoro-lower-alkyl-X.

A heterocyclic radical contains especially up to 20 carbon atoms including possible substituents and is an unsaturated, partially unsaturated, or preferably saturated monocyclic radical having from 4 or 8 ring members and from 1 to 4, especially from 1 to 3, and most preferably 1 or 2 heteroatoms which are preferably selected from nitrogen, oxygen and sulfur, or a bi- or tri-cyclic radical wherein, for example, one or two benzene radicals are annellated (fused) to the mentioned monocyclic radical. The heterocyclic radical is optionally substituted by one or more radicals such as e.g. unsubstituted or substituted lower alkyl.

In heterocyclyl-NH— and heterocyclyl-O—, the heterocyclyl moiety is as defined for a heterocyclic radical in the preceding paragraph with the proviso that it is bound to NH and O, respectively, via a carbon ring atom and is preferably piperidyl substituted by lower alkyl, such as especially 2,2,6,6-tetramethyl-piperidin-4-yl or 1-methyl-piperidin-4-yl.

A heterocyclic radical R₁ is preferably lower alkyl-piperazinyl, especially 4-lower alkyl-piperazin-1-yl.

The heterocyclic ring formed by two vicinal R₁, substituents together with the phenyl carbon atoms to which they are attached contains especially up to 20 carbon atoms including possible substituents and is an unsaturated, partially unsaturated, or saturated monocyclic radical having from 4 or 8 ring members and from 1 to 3 heteroatoms which are preferably selected from nitrogen, oxygen and sulfur. The heterocyclic ring is optionally substituted by one or more radicals such as e.g. oxo (═O), thioxo (═S), or unsubstituted or substituted lower alkyl. Preferably this ring is a thiazol, 1-oxo-thiazol or dioxol ring.

Lower alkyl substituted by a heterocyclic radical R₁ is preferably lower alkyl substituted by lower alkyl-piperazinyl, especially by 4-lower alkyl-piperazin-1-yl.

A heterocyclic radical R₄ is preferably morpholinyl, especially morpholin-4-yl, or lower alkyl-piperidyl, especially 1-lower alkyl-piperidin-4-yl.

A heterocyclic radical R₅ is preferably lower alkyl-piperazinyl, especially 4-lower alkyl-piperazin-1-yl.

A heterocyclic radical R₂ is preferably bound to the rest of the molecule of formula I via a carbon ring atom and is especially piperidyl, such as piperidin-4-yl, lower alkyl-piperidyl, such as 1-lower alkyl-piperidin-4-yl, or tetrahydro-pyranyl, such as tetrahydro-pyran-4-yl.

Etherified hydroxy is, for example, alkoxy, especially lower alkoxy, such as methoxy, ethoxy and tert-butoxy.

Etherified hydroxy R₅ is preferably lower alkoxy, especially ethoxy or tert-butoxy.

Esterified hydroxy is preferably hydroxy esterified by an organic carboxylic acid, such as alkanoic acid, and is for example lower alkanoyloxy.

Halogen is primarily fluorine, chlorine, bromine, or iodine, especially fluorine, chlorine, or bromine.

X is preferably —O—.

m is preferably from 1 to 3.

R₁ is preferably attached to the phenyl ring in the meta and/or para position.

Z is preferably attached to the phenyl ring in the meta and/or para position, most preferably the meta position.

R₄ is preferably mono- or di-substituted amino, or a heterocylic radical.

Salts are especially the pharmaceutically acceptable salts of compounds of formula I.

Such salts are formed, for example, as acid addition salts, preferably with organic or inorganic acids, from compounds of formula I with a basic nitrogen atom, especially the pharmaceutically acceptable salts.

In the presence of negatively charged radicals, such as carboxy or sulfo, salts may also be formed with bases, e.g. metal or ammonium salts, such as alkali metal or alkaline earth metal salts, or ammonium salts with ammonia or suitable organic amines, such as tertiary monoamines.

In the presence of a basic group and an acid group in the same molecule, a compound of formula I may also form internal salts.

For isolation or purification purposes it is also possible to use pharmaceutically unacceptable salts, for example picrates or perchlorates. Only the pharmaceutically acceptable salts or free compounds (if the occasion arises, in the form of pharmaceutical compositions) attain therapeutic use, and these are therefore preferred.

In view of the close relationship between the novel compounds in free form and in the form of their salts, including those salts that can be used as intermediates, for example in the purification or identification of the novel compounds, hereinbefore and hereinafter any reference to the free compounds is to be understood as referring also to the corresponding salts, as appropriate and expedient.

The compounds of formula I are potent inhibitors of the tyrosine kinase activity of the Insulin-like growth factor I receptor (IGF-IR) and inhibit IGF-IR-dependent cell. The compounds of formula I permit, for example, an unexpected new therapeutic approach, especially for diseases in the treatment of which, and also for the prevention of which, an inhibition of the IGF-IR tyrosine kinase and/or of the IGF-1R-dependent cell proliferation shows beneficial effects. Such diseases include proliferative diseases, such as tumours, like for example breast, renal, prostate, colorectal, thyroid, ovarian, pancreas, neuronal, lung, uterine and gastro-intestinal tumours as well as osteosarcomas and melanomas.

Compounds of formula I are also useful for preventing or combating graft vessel diseases, e.g. allo- or xenotransplant vasculopathies, e.g. graft vessel atherosclerosis or chronic graft rejection, e.g. in a transplant of organ, tissue or cells, e.g. heart, lung, combined heart-lung, liver, kidney or pancreatic transplants (e.g. pancreatic islet cells), or for preventing or treating vein graft stenosis, restenosis and/or vascular occlusion following vascular injury, e.g. caused by catherization procedures or vascular scraping procedures such as percutaneous transluminal angioplasty, laser treatment or other invasive procedures which disrupt the integrity of the vascular intima or endothelium.

The compounds according to the invention can be used both alone and in combination with other pharmacologically active compounds, for example together with inhibitors of the enzymes of polyamine synthesis, inhibitors of protein kinase C, inhibitors of other tyrosine kinases, cytokines, negative growth regulators, for example TGF-β or IFN-β, aromatase inhibitors, antioestrogens and/or cytostatic drugs.

With the groups of preferred compounds of formula I mentioned hereinafter, definitions of substituents from the general definitions mentioned hereinbefore may reasonably be used, for example, to replace more general definitions with more specific definitions or especially with definitions characterized as being preferred.

The present invention provides a compound of formula I

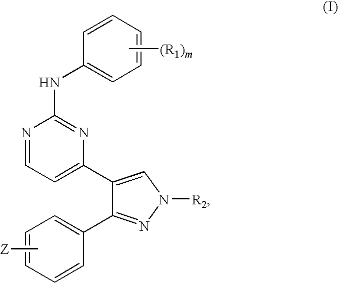

wherein

m is from 1 to 5;

R₁ is lower alkyl-sulfonyl; unsubstituted, mono- or di-substituted amino-sulfonyl; unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;

or two vicinal R₁ substituents together with the phenyl carbon atoms to which they are attached form a heterocyclic ring;

R₂ is hydrogen, unsubstituted or substituted lower alkyl or a heterocyclic radical; and

Z is benzyloxy;

or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

Preferably, the compound is a compound of the above of formula Ib

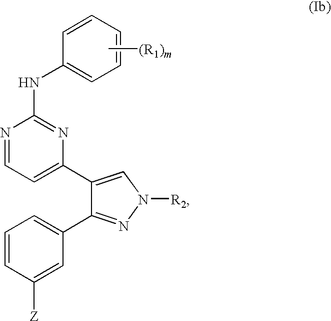

wherein

m is from 1 to 5;

R₁ is lower alkyl-sulfonyl; unsubstituted, mono- or di-substituted amino-sulfonyl;

unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;

or two vicinal R₁ substituents together with the phenyl carbon atoms to which they are attached form a heterocyclic ring;

R₂ is hydrogen, unsubstituted or substituted lower alkyl or a heterocyclic radical; and

Z is benzyloxy;

or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

Preferably, R1 is a heterocyclic radical; lower alkyl substituted by mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein

R₄ is mono- or di-substituted amino, or a heterocyclic radical, and X is —S— or —O—; or a radical R₅—C(═O)—, wherein R₆ is unsubstituted, mono- or di-substituted amino, or a heterocyclic radical; m is 1;

R2 is hydrogen;

or a or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

More preferably, R1 is a lower alkyl substituted by a di-lower alkyl substituted amino, an alkyl substituted 5- or 6-membered heterocyclyl —NH—, heterocyclyl-NH— wherein heterocyclyl is bound to NH via a carbon ring atom; a radical R₄-lower alkyl-O—, wherein R₄ is unsubstituted or di-substituted amino; or a radical R₅—C(═O)—, wherein R₅ is unsubstituted, mono- or di-substituted amino, or a heterocyclic radical; m is 1;

R2 is hydrogen;

or a or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

More preferably still, R₁ is a lower alkyl substituted by a di-lower alkyl substituted amino, or a lower alkyl-substituted piperazinyl, or a pyrrolidine; piperidinyl wherein piperidinyl is bound to NH via a carbon ring atom; a radical R₄— lower alkyl-O—, wherein R₄ is amino di-substituted by lower alkyl; or R₅—C(═O)—, wherein R₅ is a lower alkyl-substituted piperazinyl;

m is 1;

R2 is hydrogen;

or a or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

Even more preferably, the compound is chosen from the group consisting of;
{4-[3-(3-Benzyloxy-phenyl)-1H-pyrazol-4-yl]-pyrimidin-2-yl}-(4-pyrrolidin-1-ylmethyl-phenyl)-amine;
{4-[3-(3-Benzyloxy-phenyl)-1H-pyrazol-4-yl]-pyrimidin-2-yl}-(4-dimethyl aminomethyl-phenyl)-amine;
(4-{4-[3-(3-Benzyloxy-phenyl)-1H-pyrazol-4-yl]-pyrimidin-2-ylamino}-phenyl)-(4-methyl-piperazin-1-yl)-methanone;
{4-[3-(3-Benzyloxy-phenyl)-1H-pyrazol-4-yl]-pyrimidin-2-yl}-[4-(4-methyl-piperazin-1-ylmethyl)-phenyl]-amine; and
4-{4-[3-(3-Benzyloxy-phenyl)-1H-pyrazol-4-yl]-pyrimidin-2-ylamino}-N-(2,2,6,6-tetramethyl-piperidin-4-yl)-benzamide.

R₁ is preferably lower alkyl substituted by amino, lower alkyl substituted by a heterocyclic radical or R₅—C(O)—.

More preferably, R₁ is lower alkyl substituted by amino.

Even more preferably, R₁ is lower alkyl substituted by a heterocyclic radical.

Preferred is a compound according to the above, in which the alkyl portion is methylene and the heterocyclic radical is a five or six membered ring containing one or two nitrogens and is unsubstituted or substituted on one or more carbon atoms by a lower alkyl group.

R₁ is preferably R₅—C(O)—.

R₅ is preferably substituted amino or a heterocyclic radical, wherein the heterocyclic radical is a five or six membered ring containing one or two nitrogens and is unsubstituted or substituted on one or more carbon atoms by a lower alkyl group.

R₂ is preferably H.

A compound wherein m is 1 is preferred.

More preferred is a compound according to the above for medical use.

In one aspect, the present invention provides a compound according to formula I

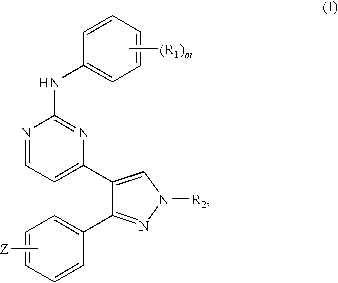

wherein

m is from 1 to 5;

R₁ is lower alkyl-sulfonyl; unsubstituted, mono- or di-substituted amino-sulfonyl; unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;

or two vicinal R₁ substituents together with the phenyl carbon atoms to which they are attached form a heterocyclic ring;

R₂ is hydrogen, unsubstituted or substituted lower alkyl or a heterocyclic radical; and

Z is benzyloxy;

or a salt of the said compounds, for medical use.

More preferred is the use of a compound according to the above for the manufacture of a medicament to be used in the treatment of a proliferative disease.

Even more preferred is the use of a compound according to the above, in which the disease is chosen form the group consisting of; tumours, for example breast, renal, prostate, colorectal, thyroid, ovarian, pancreas, neuronal, lung, uterine and gastro-intestinal tumours as well as osteosarcomas and melanomas.

In another aspect there is provided the use of a compound according to the above for the manufacture of a medicament to be used in the treatment of a graft vessel disease, or for preventing or treating vein graft stenosis, restenosis and/or vascular occlusion following vascular injury.

In another aspect there is provided a method of treating a disease which responds to inhibition of IGF-1R in a mammal, which comprises administering to the mammal an effective IGF-1R inhibiting amount of a compound of formula Ia

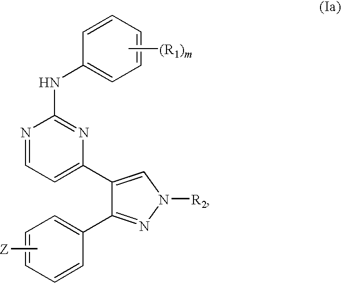

wherein

m is from 1 to 5;

R₁ is lower alkyl-sulfonyl; unsubstituted, mono- or di-substituted amino-sulfonyl;

unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;

or two vicinal R₁ substituents together with the phenyl carbon atoms to which they are attached form a heterocyclic ring;

R₂ is hydrogen, unsubstituted or substituted lower alkyl or a heterocyclic radical; and

Z is benzyloxy;

or a pharmaceutically acceptable salt thereof.

Preferably, the method according to the above comprises administering to the mammal an effective IGF-1R inhibiting amount of a compound of formula Ib

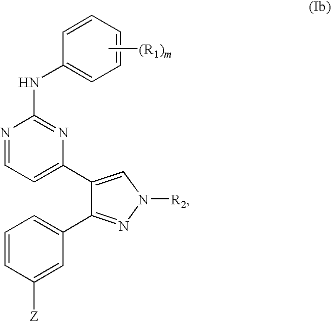

wherein

m is from 1 to 5;

R₁ is lower alkyl-sulfonyl; unsubstituted, mono- or di-substituted amino-sulfonyl;

unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is a —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;

or two vicinal R₁ substituents together with the phenyl carbon atoms to which they are attached form a heterocyclic ring;

R₂ is hydrogen, unsubstituted or substituted lower alkyl or a heterocyclic radical; and

Z is benzyloxy;

or a pharmaceutically acceptable salt thereof.

More preferably, there is provided a use of a compound according to any one of claims 1-14 for the preparation of a pharmaceutical composition for the therapeutic and/or prophylactic management of a disease that responds to inhibition of IGF-1R.

In yet another aspect, there is provided a pharmaceutical composition which comprises a pharmaceutically effective amount of a compound of any one of claims 1-14 and a pharmaceutically acceptable carrier.

The pharmaceutical composition preferably comprises a pharmaceutically effective amount of a compound of any one of claims 1-14, together with inhibitors of the enzymes of polyamine synthesis, inhibitors of protein kinase C, inhibitors of other tyrosine kinases, cytokines, negative growth regulators, for example TGF-β or IFN-β, aromatase inhibitors, antioestrogens and/or cytostatic drugs; and a pharmaceutically acceptable carrier.

The compounds of this invention or salts thereof are prepared in accordance with processes known per se, though not previously described for the manufacture of the compounds of the formula I, especially whereby

a) a compound of formula II

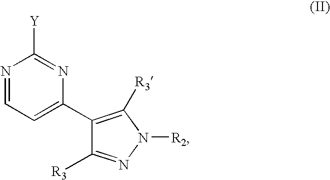

wherein Y is a leaving group such as halogen, —S(═O)—CH₃ or —S(O₂)—CH₃ and R₂, R₃ and R₃′ have the meanings as defined for a compound of formula I, is reacted with a compound of formula III

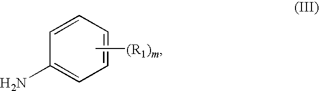

wherein m and R₁ have the meanings as defined for a compound of formula I;

b) in order to prepare a compound of formula I, wherein R₁ is a radical R₅—C(═O)— in which R₅ is mono- or di-substituted amino or a heterocyclic radical that is bound to the carbonyl moiety via a nitrogen ring atom, a compound of formula IV

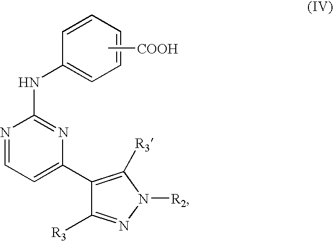

wherein R₂, R₃ and R₃′ have the meanings as defined for a compound of formula I, or a reactive carboxylic acid derivative thereof, is reacted with a mono- or di-substituted amine or a heterocyclic radical containing at least one nitrogen ring atom to which a hydrogen is bound, respectively; or

c) in order to prepare a compound of formula I, wherein R₂ is unsubstituted or substituted lower alkyl or a heterocyclic radical, a compound of formula I, wherein R₂ is hydrogen, is reacted with a compound of the formula R₂—OH, wherein R₂ is unsubstituted or substituted lower alkyl or a heterocyclic radical wherein the substituted lower alkyl or the heterocyclic radical is attached to the hydroxy group of R₂—OH via a carbon atom of the lower alkyl moiety or via a carbon ring atom of the heterocyclic radical, respectively;

whereby functional groups which are present in the starting compounds of processes a) to c) and are not intended to take part in the reaction, are present in protected form if necessary, and protecting groups that are present are cleaved, whereby the said starting compounds may also exist in the form of salts provided that a salt-forming group is present and a reaction in salt form is possible;

and, if so desired, a compound of formula I thus obtained is converted into another compound of formula I, a free compound of formula I is converted into a salt, an obtained salt of a compound of formula I is converted into the free compound or another salt, and/or a mixture of isomeric compounds of formula I is separated into the individual isomers.

Description of the Process Variants:

Regarding Process a):

The reaction between a compound of formula II, wherein Y is halogen, and a compound of formula III preferably takes place in a suitable inert solvent such as dioxane, in the presence of an acid such as HCl, at elevated temperature, preferably at around 100° C. In a compound of formula II wherein Y is halogen, halogen is preferably chloro or bromo, especially chloro.

The reaction between a compound of formula II, wherein Y is —S(O₂)—CH₃, and a compound of formula III preferably takes place under those conditions described for the analogous procedure in Klutchko et al., Journal of Medicinal Chemistry, 1998, Vol. 41, No. 17, 3276-3292.

The reaction between a compound of formula II, wherein Y is —S(═O)—CH₃, and a compound of formula III preferably takes place in a suitable inert solvent such as 1,4-dioxane or tetrahydrofuran, in the presence of BF₃.Et₂O, at elevated temperature, preferably at around 100° C.

Regarding Process b):

Reaction b), that is, the formation of amide bonds, preferably takes place under standard conditions for the formation of peptide bonds (condensation reaction). In a reactive carboxylic acid derivative of a compound of the formula IV, the carboxyl group is either functionalized as activated ester (reactive form). The reactive carboxyl groups are, however, preferably synthesized in situ (for example making use of reagents customary in peptide chemistry, e.g. for the preparation of 1-hydroxybenzotriazole, succinimide- or N-hydroxysuccinimide esters, or in situ derivatisation with condensing agents, e.g. with carbodiimides, such as dicyclohexylcarbodiimide, with carbonylimidazole, with N-[(dimethylamino)-1H-1,2,3-triazolo[4,5-b]pyridin-1-ylmethylene]-N-methylmethanaminiumhexafluorophosphate-N-oxide (HATU); with 2-(1H-benzotriazol-1-yl)-1,1,3,3-tetramethyluroniumtetrafluoroborat (HBTU), with 2-(pyridon-1-yl)-1,1,3,3-tetramethyl-uroniumtetrafluoroborate (TPTU); or benzotriazol-1-yl-oxy-tris(dimethylamino)-phosphoniumhexafluorophosphate (BOP), or similar reagents). The condensation reaction preferably takes place in the presence of a condensing agent, especially BOP, in an aprotic polar solvent, preferably a N,N-di-(lower alkyl)-lower alkanoylamide, such as dimethylform-amide, at preferred temperatures in the range from 0 to 50° C., e.g. at room temperature.

Regarding Process c):

The reaction between a compound of formula I, wherein R₂ is hydrogen, and a compound of the formula R₂—OH preferably takes place under the Mitsunobu reaction conditions such as those described in: Mitsunobu, Oyo; Synthesis 1981, p. 1-27.

Compounds of formula I can be transformed into different compounds of formula I. Such transformations include: reduction of a carbonyl group to a methylene group as in Example 32; ether cleavage as in Example 39; oxidation of a sulfide to a sulfoxide as in Example 45; de-chlorination as in Example 104; alkylation as in Example 117.

Additional Process Steps:

In the additional process steps, carried out as desired, functional groups of the starting compounds which should not take part in the reaction may be present in unprotected form or may be protected for example by one or more protecting groups. The protecting groups are then wholly or partly removed according to one of the known methods.

Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions.

The end products of formula I may however also contain substituents that can also be used as protecting groups in starting materials for the preparation of other end products of formula I. Thus, within the scope of this text, only a readily removable group that is not a constituent of the particular desired end product of formula I is designated a “protecting group”, unless the context indicates otherwise.

General Process Conditions:

All process steps described here can be carried out under known reaction conditions, preferably under those specifically mentioned, in the absence of or usually in the presence of solvents or diluents, preferably those that are inert to the reagents used and able to dissolve them, in the absence or presence of catalysts, condensing agents or neutralizing agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to −60° C., at RT, at −20 to 40° C., at 0 to 100° C. or at the boiling point of the solvent used, under atmospheric pressure or in a closed vessel, if need be under pressure, and/or in an inert, for example an argon or nitrogen, atmosphere.

In the preferred embodiment, a compound of formula I is prepared according to the processes and process steps defined in the Examples.

The compounds of formula I, including their salts, are also obtainable in the form of hydrates, or their crystals can include for example the solvent used for crystallisation (present as solvates).

Starting Materials:

The starting materials used in the above described processes a) to c) are known, capable of being prepared according to known processes, or commercially obtainable; in particular, they can be prepared using processes as described in the Examples.

In the preparation of starting materials, existing functional groups which do not participate in the reaction should, if necessary, be protected. Preferred protecting groups, their introduction and their removal are described above or in the Examples. In place of the respective starting materials and transients, salts thereof may also be used for the reaction, provided that salt-forming groups are present and the reaction with a salt is also possible. Where the term starting materials is used hereinbefore and hereinafter, the salts thereof are always included, insofar as reasonable and possible.

A compound of formula II, wherein Y is halogen, R₂ is hydrogen or lower alkyl, and R₃ and R₃′ have the meanings as defined for a compound of formula I, can be prepared for example by reacting a compound of formula V

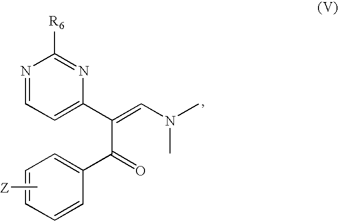

wherein R₆ is halgen, with hydrazine (H₂N—NH₂) or N-lower alkyl-hydrazine (lower alkyl-NH—NH₂), respectively, in a suitable solvent, e.g. lower alcohols, such as ethanol, preferably at around room temperature.

A compound of the formula II, wherein Y is halogen, R₂ is unsubstituted or substituted lower alkyl or a heterocyclic radical, and R₃ and R₃′ have the meanings as defined for a compound of formula I, can be prepared for example by reacting a compound of formula II, wherein Y is halogen, R₂ is hydrogen and R₃ and R₃′ have the meanings as defined for a compound of formula I, with a compound of the formula R₂—OH, wherein R₂ is unsubstituted or substituted lower alkyl or a heterocyclic radical wherein the substituted lower alkyl or the heterocyclic radical is attached to the hydroxy group of R₂—OH via a carbon atom of the lower alkyl moiety or via a carbon ring atom of the heterocyclic radical, respectively, e.g. under the Mitsunobu reaction conditions such as those described in: Mitsunobu, Oyo; Synthesis 1981, p. 1-27.

A compound of formula II, wherein Y is —S(O₂)—CH₃ and R₂, R₃ and R₃′ have the meanings as defined for a compound of formula I, can be prepared for example by reacting a compound of formula VI

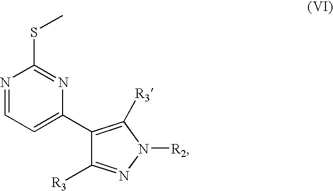

wherein R₂, R₃ and R₃′ have the meanings as defined for a compound of formula I, with 3-chloroperoxybenzoic acid in CHCl₃, e.g. under those conditions described for the analogous procedure in Klutchko et al., Journal of Medicinal Chemistry, 1998, Vol. 41, No. 17, 3276-3292.

A compound of formula II, wherein Y is —S(═O)—CH₃ and R₂, R₃ and R₃′ have the meanings as defined for a compound of formula I, can be prepared for example by reacting a compound of formula VI with 3-chloroperoxybenzoic acid under conditions such as those described in M. P. Zawistoski, Journal of Heterocyclic Chemistry, 1991, Volume 28, p. 657-665.

A compound of formula IV, or a reactive carboxylic acid derivative thereof, wherein R₂, R₃ and R₃′ have the meanings as defined for a compound of formula I, can be prepared for example by reacting a compound of formula II, wherein Y is a leaving group such as halogen, —S(═O)—CH₃ or —S(O₂)—CH₃ and R₂, R₃ and R₃′ have the meanings as defined for a compound of formula I, with amino-benzoic acid, e.g. under conditions described for the reaction of a compound of formula II with a compound of formula III, and activate the carboxy group of benzoic acid thereafter.

A compound of formula VI, wherein R₂ is hydrogen or lower alkyl and R₃ and R₃′ have the meanings as defined for a compound of formula I, can be prepared for example by reacting a compound of formula V, wherein R₆ is —S—CH₃ and Z has the meaning as defined for a compound of formula I, with hydrazine (H₂N—NH₂) or N-lower alkyl-hydrazine (lower alkyl-NH—NH₂), respectively, in a suitable solvent, e.g. lower alcohols, such as ethanol, preferably at around room temperature.

A compound of formula VI, wherein R₂ is unsubstituted or substituted lower alkyl or a heterocyclic radical, and R₃ and R₃′ have the meanings as defined for a compound of formula I, can be prepared for example by reacting a compound of formula VI, wherein R₂ is hydrogen and R₃ and R₃′ have the meanings as defined for a compound of formula I, with a compound of the formula R₂—OH, wherein R₂ is unsubstituted or substituted lower alkyl or a heterocyclic radical wherein the substituted lower alkyl or the heterocyclic radical is attached to the hydroxy group of R₂—OH via a carbon atom of the lower alkyl moiety or via a carbon ring atom of the heterocyclic radical, respectively, e.g. under the Mitsunobu reaction conditions such as those described in: Mitsunobu, Oyo; Synthesis 1981, p. 1-27.

A compound of formula V, wherein R₆ is halogen or —S—CH₃ and Z has the meaning as defined for a compound of formula I, can be prepared for example by reacting a compound of formula VII

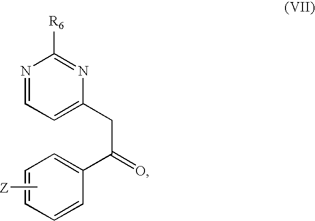

wherein R₆ is halogen or —S—CH₃, respectively, and Z has the meaning as defined for a compound of formula I, with N,N-dimethylformamid-dimethylacetal, at elevated temperature, preferably at around 100° C.

A compound of formula VII, wherein R₆ is halogen and Z has the meaning as defined for a compound of formula I, can be prepared for example by reacting a compound of formula VIII

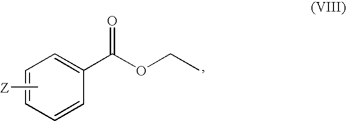

wherein Z has the meaning as defined for a compound of formula I, with a compound of formula IX

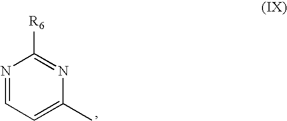

wherein R₆ is halogen, in the presence of lithiumdiisopropylamide, in a suitable organic solvent or mixture of solvents, preferably starting the reaction at reduced temperature, preferably at around −75° C., and letting it to reach room temperature.

A compound of formula VII, wherein R₆ is —S—CH₃ and Z has the meaning as defined for a compound of formula I, can be prepared for example by reacting a compound of formula X

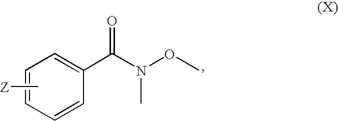

wherein Z has the meaning as defined for a compound of formula I, with a compound of formula IX, wherein R₆ is —S—CH₃, in the presence of lithiumdiisopropylamide, in a suitable organic solvent or mixture of solvents, preferably starting the reaction at reduced temperature, preferably at around −75° C., and letting it to reach room temperature.

A compound of formula X can be prepared for example by reacting a compound of formula XI

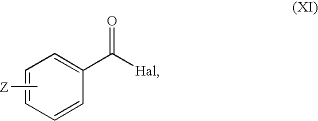

wherein Hal is halogen, such as chloro, and Z has the meaning as defined for a compound of formula I, with N—O-dimethylhydroxylamine HCl in CH₂Cl₂, e.g. under those conditions described for the analogous procedure in Nahm, Steven; Weinreb, Steven M.; Tetrahedron Lett.; 1981; 22 (39); 3815-3818.

The remaining starting materials are known, capable of being prepared according to known processes, or commercially available; or in particular, they can be prepared using processes as described in the Examples.

Pharmaceutical Compositions, Methods, and Uses:

The present invention relates also to pharmaceutical compositions that comprise a compound of formula I, or a pharmaceutically acceptable salt thereof, as active ingredient and that can be used especially in the treatment of the diseases mentioned at the beginning. Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions contain the active ingredient alone or, preferably, together with a pharmaceutically acceptable carrier. The dosage of the active ingredient depends upon the disease to be treated and upon the species, its age, weight, and individual condition, the individual pharmacokinetic data, and the mode of administration.

The present invention also relates to pro-drugs of a compound of formula I that convert in vivo to the compound of formula I as such. Any reference to a compound of formula I is therefore to be understood as referring also to the corresponding pro-drugs of the compound of formula I, as appropriate and expedient.

The invention relates also to compounds of formula I, or a pharmaceutically acceptable salt thereof, as such or in the form of a pharmaceutical composition, for use in a method for the prophylactic or especially therapeutic treatment of the human or animal body, to a process for the preparation thereof (especially in the form of compositions for the treatment of tumours) and to a method of treating proliferative diseases, primarily tumour diseases, especially those mentioned above.

The invention relates also to processes and to the use of compounds of formula I, or a pharmaceutically acceptable salt thereof, or especially compounds of formula Ib, or a pharmaceutically acceptable salt thereof, for the preparation of pharmaceutical compositions which comprise compounds of formula I, or a pharmaceutically acceptable salt thereof, or preferably compounds of formula Ib, or a pharmaceutically acceptable salt thereof, as active component (active ingredient).

If desired, the said pharmaceutical compositions may also contain further active components, for example cytostatics, and/or may be used in combination with known therapeutic processes, for example the administration of hormones or radiation.

Preference is given for a pharmaceutical composition which is suitable for administration to a warm-blooded animal, especially humans or commercially useful mammals suffering from a disease which responds to an inhibition IGF-1R, comprising a compound of formula I, preferably a compound of formula Ib, or a pharmaceutically acceptable salt thereof, together with at least one pharmaceutically acceptable carrier.

A pharmaceutical composition for the prophylactic or especially therapeutic management of neoplastic and other proliferative diseases of a warm-blooded animal, especially a human or a commercially useful mammal requiring such treatment, especially suffering from such a disease, comprising as active ingredient in a quantity that is prophylactically or especially therapeutically active against said diseases a compound of formula Ib, or a pharmaceutically acceptable salt thereof, is likewise preferred.

The pharmaceutical compositions comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, for example, coated and uncoated tablets, ampoules, vials, suppositories or capsules. Examples are capsules containing from about 0.05 g to about 1.0 g of active substance.

The pharmaceutical compositions of the present invention are prepared in a manner known per se, for example by means of conventional mixing, granulating, coating, dissolving or lyophilising processes.

The invention relates likewise to a process or a method for the treatment of one of the pathological conditions mentioned hereinabove, especially a disease which responds to inhibition of IGF-1R, especially a corresponding neoplastic disease.

Thus, the invention relates to a method of treating a disease which responds to inhibition of IGF-1R in a mammal, which comprises administering to the mammal an effective IGF-1R inhibiting amount of a compound of formula Ia

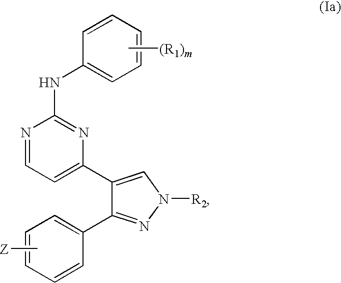

wherein

m is from 1 to 5;

R₁ is lower alkyl-sulfonyl; unsubstituted, mono- or di-substituted amino-sulfonyl;

unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;

or two vicinal R₁ substituents together with the phenyl carbon atoms to which they are attached form a heterocyclic ring;

R₂ is hydrogen, unsubstituted or substituted lower alkyl or a heterocyclic radical; and

Z is benzyloxy;

or a pharmaceutically acceptable salt thereof.

In a particular embodiment, the present invention relates to a method of treating a disease which responds to inhibition of IGF-1R in a mammal, which comprises administering to the mammal an effective IGF-1R inhibiting amount of a compound of formula Ib

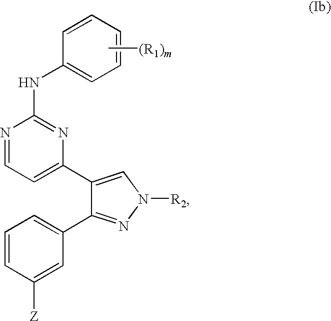

wherein

m is from 1 to 5;

R₁ is lower alkyl-sulfonyl; unsubstituted, mono- or di-substituted amino-sulfonyl;

unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is a —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;

or two vicinal R₁ substituents together with the phenyl carbon atoms to which they are attached form a heterocyclic ring;

R₂ is hydrogen, unsubstituted or substituted lower alkyl or a heterocyclic radical; and

Z is benzyloxy;

or a pharmaceutically acceptable salt thereof.

The preferences stated above for the compounds also apply to methods of using the compounds.

The compounds of formula Ia or Ib, or pharmaceutically acceptable salts thereof, can be administered as such or in the form of pharmaceutical compositions, prophylactically or therapeutically, preferably in an amount effective against the said diseases, to a warm-blooded animal, for example a human, requiring such treatment, the compounds especially being used in the form of pharmaceutical compositions. In the case of an individual having a bodyweight of about 70 kg the daily dose administered is from approximately 0.1 g to approximately 5 g, preferably from approximately 0.5 g to approximately 2 g, of a compound of the present invention.

The present invention relates especially also to the use of a compound of formula Ia or Ib, or a pharmaceutically acceptable salt thereof, especially a compound of formula Ib which is said to be preferred, or a pharmaceutically acceptable salt thereof, as such or in the form of a pharmaceutical composition with at least one pharmaceutically acceptable carrier, for the therapeutic and also prophylactic management of one or more of the diseases mentioned hereinabove.

The present invention relates especially also to the use of a compound of formula Ia or Ib, or a pharmaceutically acceptable salt thereof, especially a compound of formula Ib which is said to be preferred, or a pharmaceutically acceptable salt thereof, for the preparation of a pharmaceutical composition for the therapeutic and also prophylactic management of one or more of the diseases mentioned hereinabove, especially a neoplastic disease, in particular a disease that responds to inhibition of IGF-1R.

A compound of formula I may also be used to advantage in combination with other antiproliferative agents. Such antiproliferative agents include, but are not limited to aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, mTOR inhibitors, antineoplastic antimetabolites, platin compounds, compounds decreasing the protein kinase activity and further anti-angiogenic compounds, gonadorelin agonists, anti-androgens, bengamides, bisphosphonates, antiproliferative antibodies and temozolomide (TEMODAL®).

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETEN™.

A combination of the invention comprising an antineoplastic agent which is an aromatase inhibitor is particularly useful for the treatment of hormone receptor positive breast tumours.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™ Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “histone deacetylase inhibitors” relates to compounds which inhibit the histone deacetylase and which possess antiproliferative activity.

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enyzme (COX-2) and which possess antiproliferative activity such as celecoxib (Celebrex®), rofecoxib (Vioxx®) and lumiracoxib (COX189).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “antineoplastic antimetabolites” includes, but is not limited to 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719.

The term “platin compounds” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity and further anti-angiogenic compounds” as used herein includes, but is not limited to compounds which decrease the activity of e.g. the Vascular Endothelial Growth Factor (VEGF), the Epidermal Growth Factor (EGF), c-Src, protein kinase C, Platelet-derived Growth Factor (PDGF), Bcr-Abl tyrosine kinase, c-kit, Flt-3 and Cyclin-dependent kinases (CDKs), and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula I), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin™, described by M. S. O'Reilly et al, Cell 79, 1994, 315-328; and Endostatin™, described by M. S. O'Reilly et al, Cell 88, 1997, 277-285; compounds which decrease the activity of EGF are especially compounds which inhibit the EGF receptor, especially the tyrosine kinase activity of the EGF receptor, and compounds binding to EGF, and are in particular those compounds generically and specifically disclosed in WO 97/02266 (describing compounds of formula IV), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980;

compounds which decrease the activity of c-Src include, but are not limited to, compounds inhibiting the c-Src protein tyrosine kinase activity as defined below and to SH2 interaction inhibitors such as those disclosed in WO97/07131 and WO97/08193;

compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706;

compounds which decreases the activity of the protein kinase C are especially those staurosporine derivatives disclosed in EP 0 296 110 (pharmaceutical preparation described in WO 00/48571) which compounds are protein kinase C inhibitors;

further specific compounds that decrease protein kinase activity and which may also be used in combination with the compounds of the present invention are Imatinib (Gleevec®/Glivec®), PKC412, Iressa™ (ZD1839), PKI166, PTK787, ZD6474, GW2016, CHIR-200131, CEP-7055/CEP-5214, CP-547632 and KRN-633; anti-angiogenic compounds having another mechanism of action than decreasing the protein kinase activity include, but are not limited to e.g. thalidomide (THALOMID), celecoxib (Celebrex), SU5416 and ZD6126.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™. Abarelix can be formulated, eg. as disclosed in U.S. Pat. No. 5,843,901.

The term “anti-androgens” as used herein includes, but is not limited to bicalutamide (CASODEX™), which can be formulated, e.g. as disclosed in U.S. Pat. No. 4,636,505.

The term “bengamides” relates to bengamides and derivatives thereof having antiproliferative properties.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

The term “antiproliferative antibodies” as used herein includes, but is not limited to trastuzumab (Herceptin™), Trastuzumab-DM1, erlotinib (Tarceva™), bevacizumab (Avastin™), rituximab (Rituxan®), PRO64553 (anti-CD40) and 2C4 Antibody.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of formula I, can be prepared and administered as described in the art such as in the documents cited above.

The efficacy of the compounds of the invention as inhibitors of IGF-IR tyrosine kinase activity can be demonstrated using a cellular “Capture ELISA”. In this assay the activity of the compounds of the invention against Insulin-like growth factor I (IGF-1) induced autophosphorylation of the IGF-IR is determined. The assay is conducted as follows: For the assay NIH-3T3 mouse fibroblasts transfected with human IGF-IR cDNA (complete human IGF-IR cDNA: GenBank Acc. No. NM_{—}000875), prepared as described in Kato et al., J. Biol. Chem. 268, 2655-61, 1993, are used. The cells which overexpress human IGF-IR are cultured in Dulbecco's minimal essential (DMEM) medium, containing 10% Fetal Calf Serum (FCS). For the assay 5,000 cells/well are plated on day 1 on 96-well plates (Costar #3595) in normal growth medium and incubated for 2 days at 37° C. in a standard CO₂ cell incubator. The density of the cells does not exceed 70-80% at day 3. On day 3 the medium is discarded and the cells are incubated for 24 h in minimal medium (DMEM, containing 0.5% FCS). Compounds of formula I [starting from 10 mM dimethyl sulfoxide (DMSO) stock solutions] are added to produce final concentrations of 0.01, 0.03, 0.1, 0.3, 1, 3 and 10 μM to determine the IC₅₀ value. The cells are incubated for 90 min in the presence of a compound of formula I. Thereafter the cells are stimulated with 50 μl IGF-I (final concentration of IGF-I in the well=10 ng/ml; IGF-I is obtained from Sigma; Product Code: I 3769) and incubated for 10 min at 37° C.

The medium is discarded and the cells are washed twice with PBS/O (=Phosphate-Buffered Saline without CaCl₂) and lysed for 15 min on ice with 50 μl/well RIPA-buffer [50 mM Tris.HCl, pH=7.2, 120 mM NaCl, 1 mM EDTA, 6 mM EGTA, 1% NP-40, 20 mM NaF, 1 mM benzamidine, 15 mM sodium pyrophosphate, 1 mM Phenyl methyl sulphonyl fluoride (PMSF) and 0.5 mM Na₃VO₄] and shaken for 10 min using a 96-well plate shaker (=cellular extracts).

Packard HTRF-96 black plates are coated with 50 μl IGF-IR monoclonal Antibody (mAB) (Santa Cruz; Cat. No.: SC-462) in a concentration of 5 μg/ml at 4° C. overnight. The plates are washed twice with 0.05% (v/v) Tween-20 in Phosphate-Buffered Saline (PBS) and once with nanopure H₂O. Blocking is done for 2 h at room temperature (RT) with 3% Bovine Serum Albumin (BSA) in TBS-T buffer (20 mM Tris.HCl, pH=7.6, 137 mM NaCl, 0.05% Tween-20). After blocking, the plates are washed once with nanopure H₂O.

Cellular extracts (40 μl/well) are pipetted onto the precoated Packard plates, together with 40 μl of the anti-phosphotyrosine mouse mAB PY-20 conjugated with Alkaline Phosphatase (AP) (1:1000 diluted in RIPA buffer; the antibody is obtained from Transduction Labs; Cat. No.: P11120).

After incubating the extracts and the secondary antibody for 2 h at 4° C., the extracts are discarded, the plates are washed twice with 0.05% (v/v) Tween-20 in PBS and once with nanopure water.

90 μl/well AP substrate (CDP-Star; obtained from Tropix; Cat. No.: MS100RY) are then added and the plates are incubated for 45 min at RT in the dark, followed by measuring AP activity in a Packard Top Count Microplate Scintillation Counter. The IC₅₀ values for the compounds of formula I are calculated via linear regression analysis using the GraphPad Instat program (GraphPad Software, USA). IC₅₀ values in the range of 5 nM to 1 μM, especially in the range of 5 nM to 300 nM are found.

In vivo activity in the nude mouse xenotransplant model: female BALB/c nude mice (8-12 weeks old, Novartis Animal Farm, Sissein, Switzerland) are kept under sterile conditions with water and feed ad libitum. Tumours are induced by subcutaneous injection of tumour cells (human epithelial cell line A-431; American Type Culture Collection (ATCC), Rockville, Md., USA, Catalogue Number ATCC CRL 1555; cell line from an 85-year-old woman; epidermoid carcinoma cell line) into carrier mice. The resulting tumours pass through at least three consecutive transplantations before the start of treatment. Tumour fragments (about 25 mg) are implanted subcutaneously in the left flank of the animals using a 13-gauge trocar needle under Forene® anaesthesia (Abbott, Switzerland). Treatment with the test compound is started as soon as the tumour has reached a mean volume of 100 mm³. Tumour growth is measured two to three times a week and 24 hours after the last treatment by determining the length of two perpendicular axes. The tumour volumes are calculated in accordance with published methods (see Evans et al., Brit. J. Cancer 45, 466-8, 1982). The anti-tumour efficacy is determined as the mean increase in tumour volume of the treated animals divided by the mean increase in tumour volume of the untreated animals (controls) and, after multiplication by 100, is expressed as T/C %. Tumour regression (given in %) is reported as the smallest mean tumour volume in relation to the mean tumour volume at the start of treatment. The test compound is administered daily by gavage.

As an alternative to cell line A-431, other cell lines may also be used in the same manner, for example:
the MCF-7 breast adenocarcinoma cell line (ATCC No. HTB 22; see also J. Natl. Cancer Inst. (Bethesda) 51, 1409-16, 1973); and
the DU145 prostate carcinoma cell line DU 145 (ATCC No. HTB 81; see also Cancer Res. 37, 4049-58, 1978).

On the basis of these studies, a compound of formula I according to the invention shows therapeutic efficacy especially against proliferative diseases responsive to an inhibition of the IGF-IR tyrosine kinase.

### EXAMPLES

The following Examples serve to illustrate the invention without limiting its scope.

Abbreviations
anh., anhydrous
DCM, dichloromethane
DMF, N,N-Dimethylformamide
ES-MS, electron spray-mass spectroscopy
min, minute/s
m.p., melting point
NMP, N-methyl-2-pyrrolidone
h, hour
HPLC, high-pressure liquid chromatography
r.t., room temperature
TFA, trifluoroacetic acid
THF, tetrahydrofuran
TPTU, 2-(2-pyridon-1-yl)-1,1,3,3-tetramethyluronium tetrafluoroborate

t_{R}, retention time

Buffers for analytical HPLC: A=water/0.1% TFA and B=acetonitrile/0.09% TFA.

Grad 1: linear gradient from 2% B to 100% B in 7 min and 3 min at 100% B; column: Nucleosil C₁₈ reverse phase, 250 mm×4.6 mm, particle size 5 μm, 100 Å. Flow rate: 2.0 ml/min. Detection at 210 nm.

Grad 2: linear gradient from 2% B to 100% B in 1.75 min and 0.75 min at 100% B; column: Chromolith speedROD, 50×4.6 mm. Flow rate: 3 ml/min. Detection at 215 nm.

Grad 3: linear gradient from 20% B to 100% B in 7 min and 2 min at 100% B; column: Nucleosil 100-3 C₁₈—HD reverse phase, 125×4 mm, particle size 5 μm, 100 Å. Flow rate: 1.0 ml/min. Detection at 215 nm.

### Example 1

### {4-[3-(3-Benzyloxy-phenyl)-1H-pyrazol-4-yl]-pyrimidin-2-yl}-(4-pyrrolidin-1-ylmethyl-phenyl)-amine

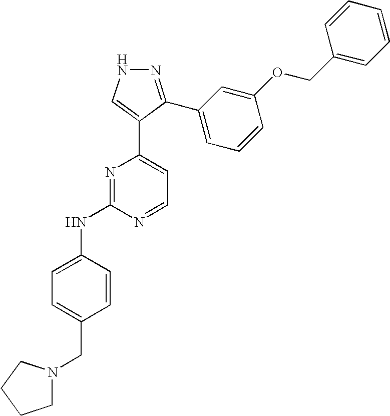

A solution of 42 mg (0.08 mmol) of (4-{4-[3-(3-benzyloxy-phenyl)-1H-pyrazol-4-yl]-pyrimidin-2-ylamino}-phenyl)-pyrrolidin-1-yl-methanone (Example 1.9.) in 2 ml of THFₐₙₕ. is added drop wise to a solution of 48 mg (1.2 mmol) of LiAlH₄ in 5 ml of THFₐₙₕ. at 0° C. and under a N₂ atmosphere. The ice-bath is removed and the mixture is stirred for 18 h at r.t. The reaction is quenched by addition of water and the suspension is extracted with ethyl acetate. The combined organic layers are washed with water, dried over Na₂SO₄, filtered and evaporated to dryness. The residue is purified by silica gel chromatography with DCM/MeOH to afford the title compound: Analytical HPLC: t_{R}=6.52 min (Grad 1); ES⁺-MS: m/eₒ=503.7.

### Example 1.1

### 3-Benzyloxy-benzoic Acid Methyl Ester

54 g (0.39 mol) of K₂CO₃ are added to a solution of 20 g (0.13 mol) of 3-hydroxy-benzoic acid methyl ester (Fluka, Switzerland) in 120 ml of DMFₐₙₕ. The mixture is stirred for 45 min at r.t. and 17.2 ml (0.14 mmol) of benzyl bromide (Merck, Dietikon, Switzerland) are added. After stirring for 50 min at r.t., water is added and the suspension is extracted with ethyl acetate. The organic phase is washed with brine, dried over Na₂SO₄, filtered and evaporated to dryness to provide the title compound: Analytical HPLC: t_{R}=1.82 min (Grad 2); ES⁺-MS: m/eₒ=243.1.

### Example 1.2

### 3-Benzyloxy-benzoic Acid

12.5 g (283 mmol) of LiOH.H₂O in 170 ml of water are added to a solution of 23.0 g (94.9 mmol) of 3-benzyloxy-benzoic acid methyl ester (Example 1.1) in 200 ml of THF. The mixture is stirred at 45° C. for 22 h. After this time, 4 N HCl is added to reach pH 1 and then the suspension is extracted with ethyl acetate. The organic phase is washed with brine, dried over Na₂SO₄, filtered and evaporated to dryness to provide the title compound: Analytical HPLC: t_{R}=1.87 min (Grad 2); ES⁻-MS: m/eₒ=227.3.

### Example 1.3

### 3-Benzyloxy-N-methoxy-N-methyl-benzamide

2.73 g (9.20 mmol) of TPTU and 5.36 ml (31.3 mmol) of diisopropylethylamine are added to a solution of 2.0 g (8.76 mmol) of 3-benzyloxy-benzoic acid (Example 1.2.) in 18 ml of NMP. After stirring the solution for 15 min at r.t., 0.94 g (9.64 mmol) of N,O-dimethylhydroxylamine hydrochloride (Fluka, Buchs, Switzerland) are added and the solution is stirred for 18 h. Ethyl acetate is added and the mixture is washed with 5% NaHCO₃, 10% ascorbic acid, water and brine. The organic phase is dried over Na₂SO₄, filtered and evaporated to dryness to provide the title compound: Analytical HPLC: t_{R}=1.75 min (Grad 2); ES⁺-MS: m/eₒ=272.3.

### Example 1.4

### 2-Chloro-4-methyl-pyrimidine

200 g (227 mol) of 2,4-dichloro-6-methylpyrimidine (Aldrich, Buchs, Switzerland) are suspended in 2 l of water/ethanol (1:1, v/v) and heated at 50° C. under stirring. Upon dissolution, 331.3 g (5.07 mol) of zinc dust (Fluka, Buchs, Switzerland) are added, followed by 10 crystals of iodine. After stirring for 20 h at 50° C., the suspension is filtered over HYFLO (Hyflo Super Cel®; Fluka, Buchs, Switzerland) Water is added to the filtrate and the mixture is extracted with tert-butylmethyl ether. The organic layer is washed with brine, dried over Na₂SO₄, filtered and evaporated to dryness to provide the title compound: Analytical HPLC: t_{R}=2.92 min (Grad 3); m.p.: 44-47° C.

### Example 1.5

### 1-(3-Benzyloxy-phenyl)-2-(2-chloro-pyrimidin-4-yl)-ethanone/1-(3-benzyloxy-phenyl)-2-(2-chloro-pyrimidin-4-yl)-ethanol

4.72 ml of a 1.6 M solution of n-butyllithium in hexane are added to a solution of 1.08 ml of diisopropylethylamine (7.56 mmol) in 4.8 ml of THFₐₙₕ. at −10° C. and under N₂ atmosphere. The solution is cooled at −70° C. and then 900 mg (6.93 mmol) of 2-chloro-4-methyl-pyrimidine (Example 1.4.) dissolved in 3 ml of THFₐₙₕ. are added drop wise. After stirring the mixture for 2 h min at −70° C., a solution of 1.71 g (6.3 mmol) of 3-benzyloxy-N-methoxy-N-methyl-benzamide (Example 1.3.) in 3 ml of THFₐₙₕ. is added. The mixture is stirred for 18 h reaching slowly r.t. The reaction is quenched with water and ethyl acetate is added. The organic phase is washed with 5% NaHCO₃, water, brine, dried over Na₂SO₄, filtered and evaporated to dryness. The residue is purified by silica gel chromatography with DCM to afford the title compound: ES⁺-MS: m/eₒ=339.4, 341.4.

### Example 1.6

### 1-(3-Benzyloxy-phenyl)-2-(2-chloro-pyrimidin-4-yl)-3-dimethylamino-propenone

5.15 g (15.2 mmol) of 1-(3-benzyloxy-phenyl)-2-(2-chloro-pyrimidin-4-yl)-ethanone (Example 1.5.) are added to 51 ml of N,N-dimethylformamide dimethyl acetal (Fluka, Buchs, Switzerland) and the mixture is heated at 80° C. for 30 min and at r.t. for 40 min. The solution is then evaporated to dryness to provide the title compound: Analytical HPLC: t_{R}=1.78 min (Grad 2); ES⁺-MS: m/eₒ=394.5.

### Example 1.7

### 4-[3-(3-Benzyloxy-phenyl)-1H-pyrazol-4-yl]-2-chloro-pyrimidine

0.5 ml (11.1 mmol) of hydrazine monohydrate, 98% are added to a solution of 4.38 g (11.1 mmol) of 1-(3-benzyloxy-phenyl)-2-(2-chloro-pyrimidin-4-yl)-3-dimethylamino-propenone (example 1.6) in 44 ml of ethanol. The solution is stirred at r.t. for 15 min and then evaporated to dryness. The residue is dissolved in ethyl acetate and the solution is extracted with 5% NaHCO₃, water, brine, dried over Na₂SO₄, filtered and evaporated to dryness to provide the title compound: Analytical HPLC: t_{R}=1.79 min (Grad 2); ES⁺-MS: m/eₒ=363.1.

### Example 1.8

### 4-{4-[3-(3-benzyloxy-phenyl)-1H-pyrazol-4-yl]-pyrimidin-2-ylamino}-benzoic Acid

800 mg (2.2 mmol) of 4-[3-(3-benzyloxy-phenyl)-1H-pyrazol-4-yl]-2-chloro-pyrimidine (Example 1.7.) and 370 mg (2.64 mmol) of 4-amino-benzoic acid (Fluka, Buchs, Switzerland) are dissolved in 6 ml of dioxane/isopropanol (1:1, v/v). The solution is heated at 180° C. for 10 min in a microwave oven (Emrys Optimizer, Personalchemistry, Uppsala, Sweden). Ethyl acetate is added and the solution is washed with brine, dried over MgSO₄, filtered and evaporated to dryness. The residue is purified by silica gel chromatography with DCM/MeOH to afford the title compound: Analytical HPLC: t_{R}=7.02 min (Grad 1); ES⁺-MS: m/eₒ=464.6.

### Example 1.9

### (4-{4-[3-(3-Benzyloxy-phenyl)-1H-pyrazol-4-yl]-pyrimidin-2-ylamino}-phenyl)-pyrrolidin-1-yl-methanone

66 mg (0.22 mmol) of 2-(2-pyridon-1-yl)-1,1,3,3-tetramethyluroniumtetrafluoroborate and 72 μl (0.42 mmol) of diisopropylethylamine are added to a solution of 93 mg (0.2 mmol) of 4-{4-[3-(3-benzyloxy-phenyl)-1H-pyrazol-4-yl]-pyrimidin-2-ylamino}-benzoic acid in 2 ml of N,N-dimethyl-acetamide. After stirring for 5 min at r.t., 33 μl (0.4 mmol) of pyrrolidine are added and the solution is stirred at r.t. for 15 min. Ethyl acetate is added and the solution is washed with brine, dried over MgSO₄, filtered and evaporated to dryness. The residue is purified by silica gel chromatography with DCM/MeOH to afford the title compound: Analytical HPLC: t_{R}=7.24 min (Grad 1); ES⁺-MS: m/eₒ=517.1.

### Example 2

### {4-[3-(3-Benzyloxy-phenyl)-1H-pyrazol-4-yl]-pyrimidin-2-yl}-(4-dimethyl aminomethyl-phenyl)-amine

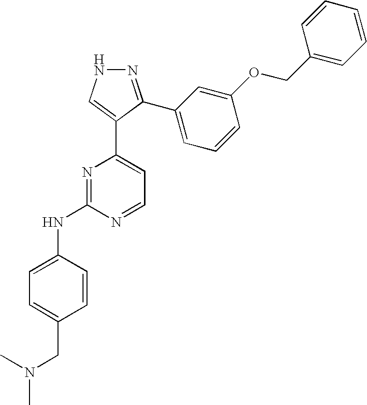

The title compound is prepared as described in Example I using dimethylamine in step 1.&. Title compound: Analytical HPLC: t_{R}=6.38 min (Grad 1); ES⁺-MS: m/eₒ=477.5.

### Example 3

### (4-{4-[3-(3-Benzyloxy-phenyl)-1H-pyrazol-4-yl]-pyrimidin-2-ylamino}-phenyl)-(4-methyl-piperazin-1-yl)-methanone

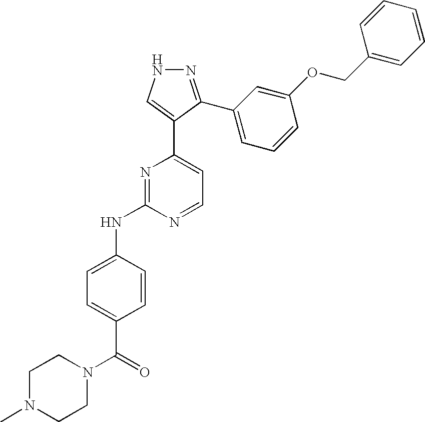

The title compound is prepared as described in Example 1 using 1-methyl-piperazine in step 1.9. Title compound: Analytical HPLC: t_{R}=6.17 min (Grad 1); ES⁺-MS: m/eₒ=546.4.

### Example 4

### {4-[3-(3-Benzyloxy-phenyl)-1H-pyrazol-4-yl]-pyrimidin-2-yl}-[4-(4-methyl-piperazin-1-ylmethyl)-phenyl]-amine

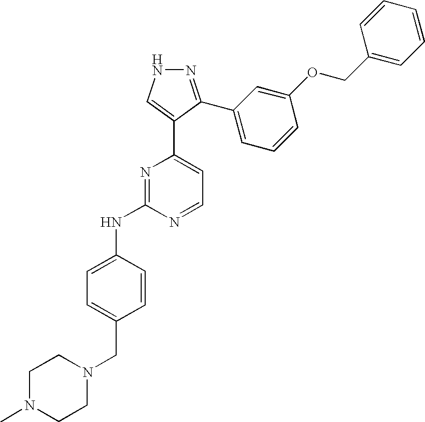

The title compound is prepared as described in Example I using 1-methyl-piperazine in step 1.9. Title compound: Analytical HPLC: t_{R}=5.98 min (Grad 1); ES⁺-MS: m/eₒ=532.7.

### Example 5

### 4-{4-[3-(3-Benzyloxy-phenyl)-1H-pyrazol-4-yl]-pyrimidin-2-ylamino}-N-(2,2,6,6-tetramethyl-piperidin-4-yl)-benzamide

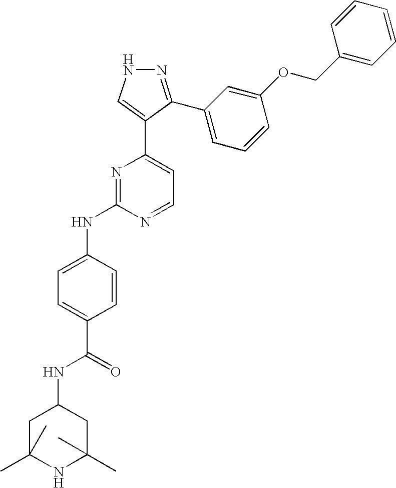

The title compound is prepared as described in Example 1 using 2,2,6,6-tetramethyl-piperidin-4-ylamine in step 1.9. Title compound: Analytical HPLC: t_{R}=6.46 min (Grad 1); ES⁺-MS: m/eₒ=602.5.

Each of the compounds of Examples 1-5 shows an inhibition of IGF-IR in the range from 70 to 96 percent at a concentration of 10 microM.

### Example 6

### {4-[3-(4-Benzyloxy-phenyl)-1H-pyrazol-4-yl]-pyrimidin-2-yl}-[4-(2-dimethylamino-ethoxy)-phenyl]-amine

The title compound is prepared as described in Example I using ethyl-4-(benzyloxy)benzoate (MAYBRIDGE 03-1741) and 4-(2-dimethylamino-ethoxy)-phenylamine, which is prepared in 3 steps from p-nitrophenol:

Step A: To the solution of 27.83 g (0.2 Mol) of 4-nitro-phenol (Fluka 73560) in 420 mL of acetone is added 55.28 g (0.4 Mol) of potassium carbonate, 143.42 g (1 Mol) of 1-bromo-2-chloro-ethane, 0.55 g (0.0033 Mol) of potassium iodide and 0.28 g (0.00087 Mol) of tetrabutyl-ammonium bromide (Fluka 86860). The resulting suspension is refluxed for 67 h. After removing the solvent under reduced pressure, the residue is taken up into ethyl acetate and washed with water. The combined organic layers are dried (Na₂SO₄), filtered and concentrated under reduced pressure. After trituration of the residue with ligroin, the crystals are filtered off to obtain 1-(2-chloro-ethoxy)-4-nitro-benzene.

Step B: 36 g (0.178 Mol) of 1-(2-Chloro-ethoxy)-4-nitro-benzene is dissolved in 360 mL of ethanol and subjected to catalytic hydration at rt using PtO₂ (1.5 g) as catalyst. The resulting suspension is diluted with CH₂Cl₂, filtered, and concentrated to approx. 150 mL. After cooling to 0° C. the crystals are filtered off, washed and dried at 60° C. under vacuum to obtain 4-(2-chloro-ethoxy)-phenylamine. Title compound: m.p.: 87-91° C.; ES-MS: 172 [M+H]⁺; single peak at t_{R}=2.73 min (System 1).

Step C, 11.15 g (0.065 Mol) of 4-(2-Chloro-ethoxy)-phenylamine is suspended in 150 mL (1.18 Mol) of dimethylamine (40% in water; Fluka 38940) and heated under stirring in a steel pressure reactor at 4 bar for 21 h. After cooling the reaction mixture is diluted with 150 mL of 2N NaOH and extracted with ethyl acetate. The combined organic layers are washed with water, dried (Na₂SO₄), filtered and evaporated under reduced pressure to obtain 4-(2-dimethylamino-ethoxy)-phenylamine. Title compound: ES-MS: 181 [M+H]⁺; single peak at t_{R}=1.10 min (System 1).

The compound of Example 6 shows inhibition of IGF-1R by the methods described above.

### Tablets 1 Comprising Compounds of the Formula (I)

Tablets, comprising, as active ingredient, 50 mg of any one of the compounds of formula (I) mentioned in the preceding Examples 1-6 of the following composition are prepared using routine methods:

| Composition: | | |
|---|---|---|
| Active Ingredient | 50 | mg |
| Wheat starch | 60 | mg |
| Lactose | 50 | mg |
| Colloidal silica | 5 | mg |
| Talcum | 9 | mg |
| Magnesium stearate | 1 | mg |
| | 175 | mg |

Manufacture: The active ingredient is combined with part of the wheat starch, the lactose and the colloidal silica and the mixture pressed through a sieve. A further part of the wheat starch is mixed with the 5-fold amount of water on a water bath to form a paste and the mixture made first is kneaded with this paste until a weakly plastic mass is formed.

The dry granules are pressed through a sieve having a mesh size of 3 mm, mixed with a pre-sieved mixture (1 mm sieve) of the remaining corn starch, magnesium stearate and talcum and compressed to form slightly biconvex tablets.

### Example 8

### Tablets 2 Comprising Compounds of the Formula (I)

Tablets, comprising, as active ingredient, 100 mg of any one of the compounds of formula (I) of Examples 1-6 are prepared with the following composition, following standard procedures:

| Composition: | | |
|---|---|---|
| Active Ingredient | 100 | mg |
| Crystalline lactose | 240 | mg |
| Avicel | 80 | mg |
| PVPPXL | 20 | mg |
| Aerosil | 2 | mg |
| Magnesium stearate | 5 | mg |
| | 447 | mg |

Manufacture: The active ingredient is mixed with the carrier materials and compressed by means of a tabletting machine (Korsch EKO, Stempeldurchmesser 10 mm).

### Example 9

### Capsules

Capsules, comprising, as active ingredient, 100 mg of any one of the compounds of formula (I) given in Examples 1-6, of the following composition are prepared according to standard procedures:

| Composition: | | |
|---|---|---|
| Active Ingredient | 100 | mg |
| Avicel | 200 | mg |
| PVPPXL | 15 | mg |
| Aerosil | 2 | mg |
| Magnesium stearate | 1.5 | mg |
| | 318.5 | mg |

Manufacturing is done by mixing the components and filling them into hard gelatine capsules, size 1.

1: A compound of formula I 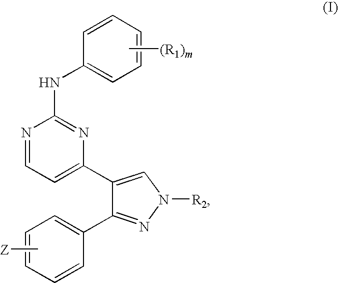
wherein
m is from 1 to 5;
R₁ is lower alkyl-sulfonyl; unsubstituted, mono- or di-substituted amino-sulfonyl;
unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;
or two vicinal R₁ substituents together with the phenyl carbon atoms to which they are attached form a heterocyclic ring;
R₂ is hydrogen, unsubstituted or substituted lower alkyl or a heterocyclic radical; and
Z is benzyloxy;
or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

2: A compound of claim 1 of formula Ib 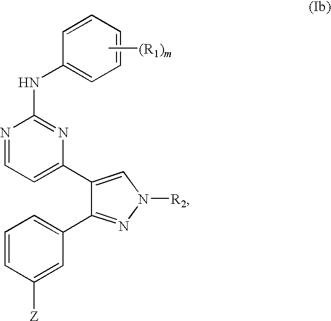
wherein
m is from 1 to 5;
R₁ is lower alkyl-sulfonyl; unsubstituted, mono- or di-substituted amino-sulfonyl;
unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;
or two vicinal R₁ substituents together with the phenyl carbon atoms to which they are attached form a heterocyclic ring;
R₂ is hydrogen, unsubstituted or substituted lower alkyl or a heterocyclic radical; and
Z is benzyloxy;
or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

3: A compound according to claim 1, in which R1 is a heterocyclic radical; lower alkyl substituted by mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is mono- or di-substituted amino, or a heterocyclic radical, and X is —S— or —O—; or a radical R₅—C(═O)—, wherein R₅ is unsubstituted, mono- or di-substituted amino, or a heterocyclic radical; m is 1;
R2 is hydrogen;
or a or a salt of the said compounds, with the proviso that the compound {4-[3-(4-Benzyloxy-phenyl)-1H-pyrazol-4-yl]-pyrimidin-2-y}-[4-(2-dimethylamino-ethoxy)-phenyl]-amine is excluded.

4: A compound according to claim 1, in which R1 is a lower alkyl substituted by a di-lower alkyl substituted amino, an alkyl substituted 5- or 6-membered heterocyclyl —NH—, heterocyclyl-NH— wherein heterocyclyl is bound to NH via a carbon ring atom; a radical R₄-lower alkyl-O—, wherein R₄ is di-substituted amino; or a radical R₅—C(═O)—, wherein R₅ is unsubstituted, mono- or di-substituted amino, or a heterocyclic radical; m is 1;
R2 is hydrogen;
or a or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

5: A compound according to claim 1, in which R₁ is a lower alkyl substituted by a di-lower alkyl substituted amino, or a C₁-C₄ alkyl-substituted piperazinyl, or a pyrrolidinyl; piperidinyl wherein piperidinyl is bound to NH via a carbon ring atom; a radical R₄— lower alkyl-O—, wherein R₄ is amino di-substituted by lower alkyl; or R₅—C(═O)—, wherein R₅ is a C₁-C₄ alkyl-substituted piperazinyl;
m is 1;
R2 is hydrogen;
or a or a salt of the said compounds, with the proviso that the compound {4-[3-(4-Benzyloxy-phenyl)-1H-pyrazol-4-yl]-pyrimidin-2-yl}-[4-(2-dimethylamino-ethoxy)-phenyl]-amine is excluded.

6: A compound chosen from the group consisting of;
{4-[3-(3-Benzyloxy-phenyl)-1H-pyrazol-4-yl]-pyrimidin-2-yl}-(4-pyrrolidin-1-ylmethyl-phenyl)-amine;
{4-[3-(3-Benzyloxy-phenyl)-1H-pyrazol-4-yl]-pyrimidin-2-yl}-(4-dimethyl aminomethyl-phenyl)-amine;
(4-{4-[3-(3-Benzyloxy-phenyl)-1H-pyrazol-4-yl]-pyrimidin-2-ylamino}-phenyl)-(4-methyl-piperazin-1-yl)-methanone;
{4-[3-(3-Benzyloxy-phenyl)-1H-pyrazol-4-yl]-pyrimidin-2-yl}-[4-(4-methyl-piperazin-1-ylmethyl)-phenyl]-amine; and
4-{4-[3-(3-Benzyloxy-phenyl)-1H-pyrazol-4-yl]-pyrimidin-2-ylamino}-N-(2,2,6,6-tetramethyl-piperidin-4-yl)-benzamide.

7: A compound of claim 2 wherein R₁ is lower alkyl substituted by amino, lower alkyl substituted by a heterocyclic radical or R₅—C(O)—.

8: A compound of claim 7 wherein R₁ is lower alkyl substituted by amino.

9: A compound of claim 7 wherein R₁ is lower alkyl substituted by a heterocyclic radical.

10: A compound of claim 9 wherein the alkyl portion is methylene and the heterocyclic radical is a five or six membered ring containing one or two nitrogens and is unsubstituted or substituted on one or more carbon atoms by a lower alkyl group.

11: A compound of claim 7 wherein R₁ is R₅—C(O)—.

12: A compound of claim 11 wherein R₅ is substituted amino or a heterocyclic radical, wherein the heterocyclic radical is a five or six membered ring containing one or two nitrogens and is unsubstituted or substituted on one or more carbon atoms by a lower alkyl group.

13: A compound of claim 7 wherein R₂ is H.

14: A compound of claim 7 wherein m is 1.

15: A compound according to formula I 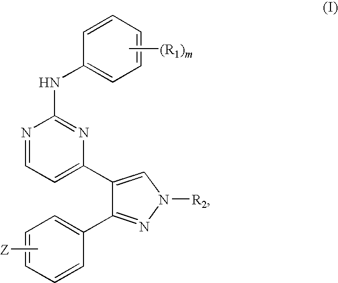
wherein
m is from 1 to 5;
R₁ is lower alkyl-sulfonyl; unsubstituted, mono- or di-substituted amino-sulfonyl;
unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;
or two vicinal R₁ substituents together with the phenyl carbon atoms to which they are attached form a heterocyclic ring;
R₂ is hydrogen, unsubstituted or substituted lower alkyl or a heterocyclic radical; and
Z is benzyloxy;
or a salt of the said compounds, for medical use.

16-17. (canceled)

18: A method according to claim 20, in which the disease is chosen form the group consisting of;
tumours, for example breast, renal, prostate, colorectal, thyroid, ovarian, pancreas, neuronal, lung, uterine and gastro-intestinal tumours as well as osteosarcomas and melanomas.

19: A method according to claim 20 wherein the disease is a graft vessel disease, or for preventing or treating vein graft stenosis, restenosis and/or vascular occlusion following vascular injury.

20: A method of treating a disease which responds to inhibition of IGF-1R in a mammal, which comprises administering to the mammal an effective IGF-1R inhibiting amount of a compound of formula Ia 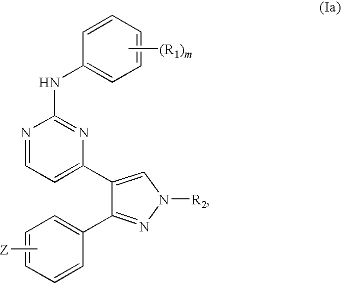
wherein
m is from 1 to 5;
R₁ is lower alkyl-sulfonyl; unsubstituted, mono- or di-substituted amino-sulfonyl;
unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;
or two vicinal R₁ substituents together with the phenyl carbon atoms to which they are attached form a heterocyclic ring;
R₂ is hydrogen, unsubstituted or substituted lower alkyl or a heterocyclic radical; and
Z is benzyloxy;
or a pharmaceutically acceptable salt thereof.

21: A method of claim 20, which comprises administering to the mammal an effective IGF-1R inhibiting amount of a compound of formula Ib 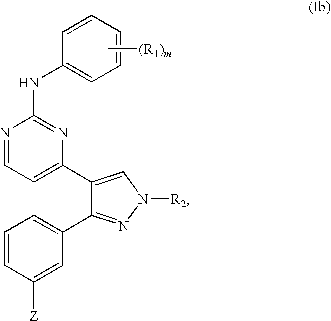
wherein
m is from 1 to 5;
R₁ is lower alkyl-sulfonyl; unsubstituted, mono- or di-substituted amino-sulfonyl;
unsubstituted, mono- or di-substituted amino; a heterocyclic radical; lower alkyl substituted by amino, mono- or di-lower alkyl substituted amino, a heterocyclic radical, heterocyclyl-NH— or heterocyclyl-O— wherein heterocyclyl is bound to NH or O via a carbon ring atom; a radical R₄-lower alkyl-X—, wherein R₄ is hydrogen, halogen, unsubstituted, mono- or di-substituted amino, or a heterocyclic radical, and X is a —S— or —O—; or a radical R₅—C(═O)—, wherein R₅ is hydrogen, unsubstituted or substituted lower alkyl, free or etherified hydroxy, unsubstituted, mono- or di-substituted amino, or a heterocyclic radical; wherein the R₁ substituents are selected independently of one another if m>1;
or two vicinal R₁ substituents together with the phenyl carbon atoms to which they are attached form a heterocyclic ring;
R₂ is hydrogen, unsubstituted or substituted lower alkyl or a heterocyclic radical; and
Z is benzyloxy;
or a pharmaceutically acceptable salt thereof.

22. (canceled)

23: A pharmaceutical composition which comprises a pharmaceutically effective amount of a compound of claim 1 and a pharmaceutically acceptable carrier.

24: A pharmaceutical composition which comprises a pharmaceutically effective amount of a compound of claim 1, together with inhibitors of the enzymes of polyamine synthesis, inhibitors of protein kinase C, inhibitors of other tyrosine kinases, cytokines, negative growth regulators, for example TGF-β or IFN-β, aromatase inhibitors, antioestrogens and/or cytostatic drugs; and a pharmaceutically acceptable carrier.

25: A pharmaceutical composition which comprises a pharmaceutically effective amount of a compound of claim 15 and a pharmaceutically acceptable carrier.

26: A pharmaceutical composition which comprises a pharmaceutically effective amount of a compound of claim 15, together with inhibitors of the enzymes of polyamine synthesis, inhibitors of protein kinase C, inhibitors of other tyrosine kinases, cytokines, negative growth regulators, for example TGF-β or IFN-β, aromatase inhibitors, antioestrogens and/or cytostatic drugs; and a pharmaceutically acceptable carrier.
